(19) United States
(12) Patent Application Publication
(10) Pub. No.: US 2006/0116745 A1
(43) Pub. Date: Jun. 1, 2006

(54) THERMOTHERAPY DEVICE

(75) Inventors: Masao HATTORI, Osaka (JP); Takahiko OISHI, Osaka (JP)

Correspondence Address: SNELL & WILMER;ONE ARIZONA CENTER, 400 EAST VAN BUREN, PHOENIX, AZ, 850040001, US

(73) Assignee: THERMEDIX CO., LTD., Hyogo (JP)

(21) Appl. No.: 11/275,927

(22) Filed: Feb. 3, 2006

Related U.S. Application Data
(62) Division of application No. 10/258,432, filed on Sep. 23, 2003.

(30) Foreign Application Priority Data
Apr. 24, 2000 (JP) 2000122061

(51) Int. Cl. A61F 7/00

(52) U.S. Cl. 607/104

(57) ABSTRACT

A thermotherapy device for treating the substantially whole body of a human being by thermotherapy. The thermotherapy device is for a human body and provided with a heat capsule (5) and a control computer (8). The heat capsule (5) is filled with a Ringer's solution, and the substantially whole body of a human being except the head can be soaked in the Ringer's solution. The control computer (8) controls the temperature of the Ringer's solution in the heat capsule (5) to control the body temperature of the substantially whole body to a predetermined treatment temperature.

### FIELD OF THE INVENTION

The present invention relates to a thermotherapy device especially for a human body.

### BACKGROUND OF THE INVENTION

From the past, it's known that cancer cells and viruses are more temperature-sensitive than healthy cells. And thermotherapy has been developed using the difference in temperature-sensitivity of these cancer cells or viruses, and healthy cells.

Thermotherapy devices used for such conventional thermotherapy have been used to locally treat specific parts of a human body using high-frequency electromagnetic radiation or radiation rays, etc.

However, since the above conventional thermotherapy devices are used to locally treat specific parts of a human body, for example, when cancer cells metastasize to different parts of the body that are difficult to find, there is the problem that thermotherapy effects are not acquired.

This invention is made in order to resolve the above problem and one purpose of this invention is to provide a thermotherapy device that can treat almost all parts of a human body.

Another purpose of this invention is to provide a thermotherapy device that can be effectively used for medical treatment, even when it is unknown whether or not cancer cells have already metastasized to different parts of the body.

The other purpose of this invention is to provide a thermotherapy device that can adjust the temperature of a human body with a high degree of accuracy, when treated by thermotherapy.

### SUMMARY OF THE INVENTION

A thermotherapy device in claim 1, which is the thermotherapy device for a human body, is equipped with a container and a control device. The container is filled up with a liquid, and capable of submerging a whole human body, except for the head, in the liquid. Also, the control device controls the temperature of the liquid in the container, and then, it controls the temperature of almost a whole human body to a predetermined temperature for therapy.

In claim 1, as above, the thermotherapy device is equipped with the container capable of submerging almost a whole human body, except for the head, in the liquid and with the control device which controls the temperature of almost a whole human body to a predetermined temperature for therapy by controlling the temperature of the liquid in the container, and then, they can easily treat almost a whole human body by thermotherapy. Thus, for example, even when it is unknown whether or not cancer cells have already metastasized to different parts of the body, it can destroy cancer cells in a whole human body, or can extinguish the proliferation of cancer cells.

A thermotherapy device in claim 2, in the construction of claim 1, when a human body is submerged in the liquid in the container, the human body is given general anesthesia. In claim 2, by giving human body general anesthesia as above, the regulation function of body temperature is depressed, and the temperature of the whole human body becomes the same to that of the surrounding atmosphere. Thus, by controlling the temperature of the environment (the temperature of the liquid in the container), the thermotherapy device can easily adjust the body temperature. As a result, it can control the temperature of a human body to a predetermined temperature for therapy.

A thermotherapy device in claim 3, in the construction of claim 1 or 2, the container includes a capsule-like container. In claim 3, the capsule-like container prevents the thermotherapy device from receiving the influence of the external atmosphere. As a result, the thermotherapy device can evenly maintain the temperature of the liquid in the container.

A thermotherapy device in claim 4, in the construction of claim 3, the capsule-like container is composed of materials of a low thermal conductivity and a high thermal effect. In claim 4, by the above construction, the thermotherapy device can easily and evenly maintain the temperature of the liquid in the container.

The thermotherapy device in claim 5, in the construction of claims from 1 to 4, the container is composed of the devices that have several outlets to infuse the liquid into the container. In claim 5, when the devices provide the liquid into the container, the liquid outflows through several outlets. Thus, the thermotherapy device with several outlets can evenly maintain the temperature of the liquid in the container more easily than that with one outlet.

A thermotherapy device in claim 6, in the construction of claims from 1 to 5, the container is equipped with a heat sensor to detect the temperature of the liquid in the container, and with the component for agitation to agitate the liquid in the container. In claim 6, by the above components, the thermotherapy device can easily detect the temperature of the liquid in the container by the thermal sensor, and evenly maintain the temperature of liquid in the container by the component for agitation.

A thermotherapy device in claim 7, in the construction of claims from 1 to 6, the container is equipped with tanks to infuse the liquid into the container. In addition, the tanks are composed of an adjusting tank to adjust the density of the liquid. In claim 7, by the above component, the thermotherapy device can easily produce the liquid of a predetermined density.

A thermotherapy device in claim 8, in the construction of claims from 1 to 7, the container is equipped with additional tanks. And the tanks contain a low-temperature tank reserving the liquid at a predetermined temperature and a high-temperature tank reserving the liquid at a higher temperature than that of the low-temperature tank and a mixture tank to get the liquid at a predetermined temperature by blending the liquid of the high-temperature tank with that of the low-temperature tank. In claim 8, by the above construction, the thermotherapy device can regulate the mixture ratio of the liquid from the low-temperature tank and the high-temperature tank in the mixture tank. Thus, the thermotherapy device can control the temperature of the liquid in the mixture tank with a high degree of accuracy.

A thermotherapy device in claim 9, in the construction of claim 7 or 8, the container contains heat sensors to detect the temperature of the liquid in the container. And in the thermotherapy device, a control computer controls the operation of the tank according to the information of the temperature detected by the heat sensor. And then, the control unit carries out feedback control so that the temperature of the liquid in the container may adjust to a predetermined setting temperature. In claim 9, by the above construction, the thermotherapy device controls the temperature of the liquid in the container with a high degree of accuracy. Thus, the thermotherapy device controls the temperature of the human body with a high degree of accuracy.

A thermotherapy device in claim 10, in the construction of claims from 1 to 9, the liquid contains the liquid of a similar osmotic pressure to that of human body fluid. In claim 10, the device of the above construction effectively prevents the liquid from penetrating through the skin of a human body when the human body is submerged in the above liquid. As a result, the device effectively prevents the skin from swelling up by submerging it in the above liquid through therapy, even after prolonged exposure.

A thermotherapy device in claim 11, in the construction of claims from 1 to 10, the liquid in the container is drained off without reuse. In claim 11, the liquid in the container without reuse effectively prevents bacteria from breeding even if bacteria outbreak occurs when the liquid is maintained at a predetermined high temperature. As a result, no problem occurs from the point of view of sanitary conditions.

A thermotherapy device in claim 12, in the construction of claims from 1 to 10, a predetermined temperature for therapy is above 37 degrees centigrade and below 43 degrees centigrade. In claim 12, the predetermined temperature for therapy is set below 43 degrees centigrade that is the thermal death point of the normal cells. Thus, the device prevents normal cells from becoming extinct and can make cancer cells etc. become extinct or can depress the breeding power of cancer cells etc.

### BRIEF DESCRIPTION OF THE DRAWINGS

FIG. 1 is the schematic view that demonstrates the whole structure of the thermotherapy device implementing this invention in some instances.

### DETAILED DESCRIPTION

Hereinafter, preferred embodiments of the present invention will be concretely described with reference to the drawings.

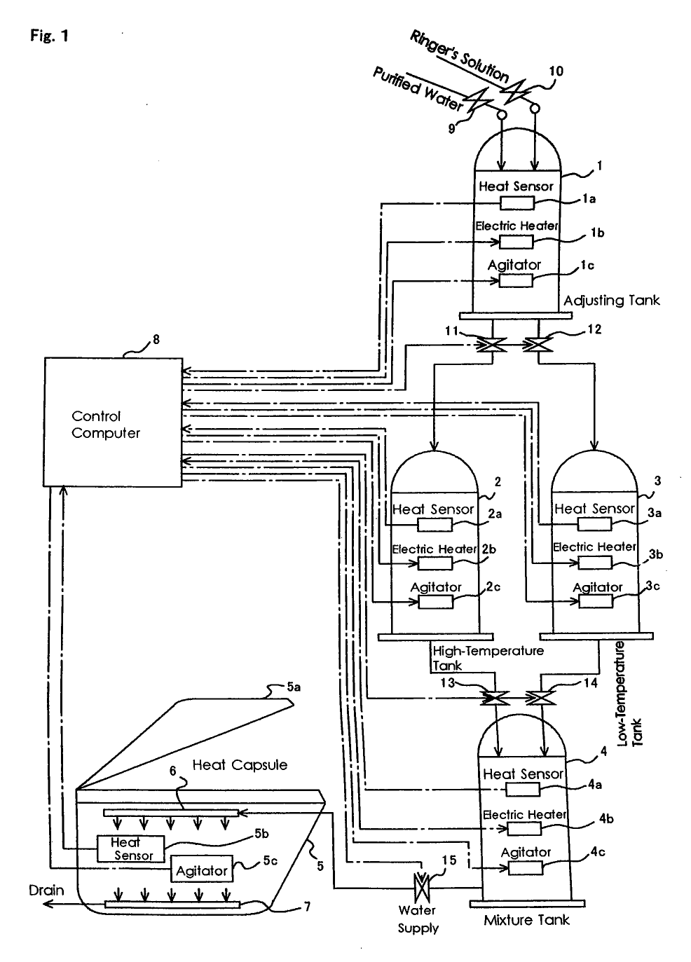

FIG. 1 shows the whole construction of a thermotherapy device in an embodiment of the prevent invention.

With reference to FIG. 1, the thermotherapy device in the present embodiment is equipped with an adjusting tank 1, a high-temperature tank 2, a low-temperature tank 3, a mixture tank 4, a heat capsule 5, an outlet part 6, a drain part 7, and a control computer 8.

The adjusting tank 1, the high-temperature tank 2, the low-temperature tank 3, and the mixture tank 4 contain heat sensors 1a, 2a, 3a and 4a to detect the temperature of the Ringer's solution in the container, electric heaters 1b, 2b, 3b and 4b to heat the Ringer's solution in each of the tanks, and agitators 1c, 2c, 3c and 4c to agitate the liquid in each tank.

The information of the temperature detected by the heat sensor is transmitted to the control computer 8. Also, both the electric heaters 1b, 2b, 3b, 4b and the agitators 1c, 2c, 3c, 4c are controlled by the control computer 8. Also, each the adjusting tank 1, the high-temperature tank 2, the low-temperature tank 3 and the mixture tank 4 has the capacity of approximately 250 liters to 300 liters.

The adjusting tank 1, by regulating valves 9 and 10, blends the Ringer's solution with the purified water at a predetermined mixture ratio. Thus the adjusting tank 1 controls the density of the Ringer's solution. This adjusting tank 1 is controlled by the control computer 8 in order that the Ringer's solution in the adjusting tank 1 adjusts to a predetermined temperature (for instance, 37 degrees centigrade).

Also, the adjusting tank 1 is connected to the high-temperature tank 2 and the low-temperature tank through the valve 11 and 12 respectively to regulate the flow rate. The high-temperature tank 2 is controlled in order that the Ringer's solution in the high-temperature tank 2 adjusts to a predetermined high temperature (for instance, 43 degrees centigrade). Also, the low-temperature tank 3 is controlled by the control computer 8 in order that the Ringer's solution in the low-temperature tank 3 adjusts to a predetermined low temperature (for instance, 37 degrees centigrade). Also, the valves 11 and 12, to regulate the flow rate, are controlled by the control signals of the control computer 8.

The high-temperature tank 2 and the low-temperature tank 3 are connected to the mixture tank 4. The valve 13, to regulate the flow rate, is set between the high-temperature tank 2 and the mixture tank 4. And the valve 14, to regulate the flow rate, is set between the low-temperature tank 3 and the mixture tank 4. The mixture tank 4 is to adjust the Ringer's solution to a predetermined temperature by blending the Ringer's solution in the high-temperature tank 2 with the Ringer's solution in the low-temperature tank 3. Also, the mixture tank 4 is controlled by the control computer 8 in order that the Ringer's solution in the mixture tank 4 is maintained at a predetermined temperature. Also, the valves 13 and 14 are controlled by the control signals of the control computer 8.

The mixture tank 4 is connected to the heat capsule 5 through the valve 15 for water supply. The valve 15 for water supply is controlled by the control signals of the control computer 8. The heat capsule 5 can be filled with about 250 liters of the Ringer's solution. Also, the heat capsule 5 is constructed in order that the whole human body except for the head can be submerged in the Ringer's solution.

Also, the heat capsule 5 contains the device of the lid 5a, the heat sensor 5b to detect the temperature of the Ringer's solution in the heat capsule 5, and the agitator 5c to agitate the Ringer's solution in the heat capsule 5. The information of the temperature detected by the heat sensor 5b is transmitted to the control computer 8. Also, the agitator 5c is controlled by the control signals of the control computer 8.

Also, the capsule 5 is constructed to have a low thermal conductivity and contains a material of high thermal effect. For example, the capsule 5 is formed with the double-layered structure of fiber-reinforced glass and a urethane foam filled between the double-layered structures. The inside of the heat capsule 5 is equipped with the outlet 6 to discharge the Ringer's solution from the mixture tank 4 into the heat capsule 5, and the drain 7 to drain the Ringer's solution in the heat capsule 5 out of the capsule. The outlet 6 has several outlet points.

The control computer 8 controls operations of the adjusting tank 1, the high-temperature tank 2, the low-temperature tank 3, the mixture tank 4, the valves 11, 12, 13 and 14 to regulate the flow rate, and the valve 15 for water supply. That is, the control computer 8 carries out-feedback control so that the temperature of the Ringer's solution in the heat capsule 5 adjusts to a predetermined setting temperature for therapy by controlling the operation of the adjusting tank 1, the high-temperature tank 2, the low-temperature tank 3 and the mixture tank 4, according to the information of the temperature of the Ringer's solution in the heat capsule 5.

Then, the operation of the thermotherapy device in the embodiment of the present invention is demonstrated.

Firstly, the thermotherapy device in the embodiment of the present invention treats cells by using the difference in the temperature-sensitivity of the cancer cells or viruses, and healthy cells. For example, it is known that the difference in the temperature-sensitivity of cancer cells and healthy cells is from 1.0 to 2.5 degrees centigrade. That is, it is known that cancer cells become extinct or lose the breeding power at the temperature from 40 to 41.5 degrees centigrade while healthy cells become extinct at about 43 degrees.

Thus, the thermotherapy device in the embodiment of the present invention, for example in the use of therapy for cancer cells, controls the temperature of the Ringer's solution in the heat capsule 5 at the range of 40 to 41.5 degrees centigrade, then, controls the temperature of a human body in the Ringer's solution at the range of 40 to 41.5 degree centigrade. By this control, with the prevention of the extinction of healthy cells, the thermotherapy device can make cancer cells extinct or lose the breeding power.

Concerning the concrete operation, firstly, a predetermined temperature for therapy is set at the control computer 8. According to this setting, the control computer 8 transmits the control signals for operation to the adjusting tank 1, the high-temperature tank 2, the low-temperature tank 3, the mixture tank 4, the flow rate regulating valves 11, 12, 13, 14 and valve 15 for water supply in order that the temperature of the Ringer's solution filled in the heat capsule 5 becomes the setting temperature for therapy. At first, a predetermined temperature for therapy is set at 37 degrees centigrade, and it is raised at the rate of about 1 degree per one hour. However, a predetermined temperature for therapy is set at the range of 37 to 43 degrees centigrade. This is the reason why a predetermined temperature for therapy set at below 43 degrees centigrade can prevent healthy cells from becoming extinct.

In this case, firstly, regulating valves 9 and 10, the thermotherapy device supplies the purified water and the Ringer's solution into the adjusting tank 1 at a predetermined mixture ratio between the water and the solution. Thus, the device produces the Ringer's solution that has the same osmotic pressure as that of the human body fluid. This Ringer's solution is produced to become a liquid with a concentration of 0.8 pH for example. Also, the control computer 8 controls the electric heater 1b and the agitator 1c in accordance with the information of the temperature from the heat sensor 1a in the adjusting tank 1. By this, it controls in order that the temperature of the Ringer's solution in the adjusting tank 1 levels off at a predetermined temperature (for instance, 37 degrees centigrade).

Then, the flow rate regulating valves 11 and 12 are controlled by the control signals from the control computer 8, and a predetermined quantity of the Ringer's solution is provided from the adjusting tank 1 into both the high-temperature tank 2 and the low-temperature tank 3.

And, the control computer 8 controls the electric heater 2b and the agitator 2c in the high-temperature tank 2 according to the information of the temperature from the heat sensor 2a in the high-temperature tank 2, and by this, it controls the temperature in order that the temperature of the Ringer's solution in the high-temperature tank 2 becomes a predetermined high temperature (for instance, 43 degrees centigrade).

Also, the control computer 8 controls the electric heater 3b and the agitator 3c in the low-temperature tank 3 according to the information of the temperature from the heat sensor 3a in the low-temperature tank 3, and by this, it controls the temperature in order that the temperature of the Ringer's solution in the low-temperature tank 3 matches a predetermined low temperature (for instance, 37 degrees centigrade).

And, the flow rate regulating valves 13 and 14 are controlled by the control signals from the control computer 8, and then, the Ringer's solution in the high-temperature tank 2 and the low-temperature tank 3 are infused into the mixture tank 4 at a predetermined mixture ratio of the Ringer's solution between the high-temperature tank 2 and the low-temperature tank 3. Thus, in the mixture tank 4, the Ringer's solution of a predetermined temperature is produced. Also, the control computer 8 controls the heat sensor 4b and the agitator 4c according to the information of the temperature from the heat sensor 4a in the mixture tank 4, and by this, it controls the Ringer's solution to maintain at the predetermined temperature.

The valve for water supply 15 is controlled by the control signal from the control computer 8, and then, a predetermined quantity of the Ringer's solution at a predetermined temperature from the mixture tank 4 is provided to the outlet 6 of the heat capsule 5. Thus, the Ringer's solution at the predetermined temperature outflows from several outlets of the discharge header 6. To outflow the Ringer's solution from several outlets in this way, the thermotherapy device can make the temperature of the Ringer's solution more uniform than those with one outlet. Also, the Ringer's solution in the heat capsule 5 is agitated with the agitator 5c according to the control signal from the control computer 8. By this way, the temperature of the Ringer's solution in the heat capsule 5 can be made more uniformly.

Also, the Ringer's solution filled in the heat capsule 5 is drained off without reuse by the overflow from the heat capsule 5 in the use of the thermotherapy device. Also, after the use of the thermotherapy device, it's drained off from the drain and thrown out without reuse.

In addition, a human body is given general anesthesia when the human body is submerged in the Ringer's solution in the heat capsule 5 at the time of thermotherapy. This general anesthesia given to the human body makes the body temperature regulating function depressed and makes the whole body temperature the same as that of the atmosphere (the temperature of the Ringer's solution in the heat capsule 5). Thus, controlling the temperature of the Ringer's solution in the heat capsule 5, the thermotherapy device can easily regulate the human body temperature. As a result, it can easily control the human body temperature to the temperature for therapy.

And, in the situation that the human body is submerged in the Ringer's solution in the heat capsule 5, therapy by the above thermotherapy device is performed at the rate of 12 hours to 24 hours per one cycle.

The thermotherapy device in the embodiment of the present invention, as above, equipped with the heat capsule 5 which can submerge almost a whole human body except for the head in the Ringer's solution and the control computer 8 to control the temperature of the almost whole human body to be a predetermined temperature for therapy by controlling the temperature of the Ringer's solution in the heat capsule 5, can easily treat almost a whole human body by thermotherapy. Thus, for example, even when it is unknown whether or not cancer cells have already metastasized to different parts of the body, the device can make cancer cells extinct through the whole body or can depress the breeding power of the cancer cells.

Also, the thermotherapy device, as above, can maintain the temperature of the Ringer's solution in the heat capsule 5 uniformly by the shape of the heat capsule 5. In addition, the device can maintain the temperature of the Ringer's solution in the heat capsule 5 more uniformly by the construction of the heat capsule 5 with materials of a low thermal conductivity and a high thermal effect.

Also, the thermotherapy device in the embodiment of the present invention, as above, equipped with the high-temperature tank 2 and the low-temperature tank 3 and the mixture tank 4 to get the Ringer's solution at a predetermined temperature with the mixture of the Ringer's solution of the high-temperature tank 2 and the low-temperature tank 3, by regulating the mixture ratio between the Ringer's solution of the high-temperature tank 2 and the low-temperature tank 3, controls the temperature of the Ringer's solution in the mixture tank 4 by 0.1 degree centigrade with a high degree of accuracy. Thus, it can control the temperature of the Ringer's solution provided into the heat capsule 5 by 0.1 degree centigrade with a high degree of accuracy.

Also, the thermotherapy device in the embodiment of the present invention, as above, equipped with the control computer 8 to carry out feedback control in order that the temperature of the Ringer's solution in the heat capsule 5 becomes a predetermined temperature for therapy, can control the temperature of the Ringer's solution in the heat capsule 5 with a high degree of accuracy. Thus, it can control the temperature of a human body submerged in the Ringer's solution with a high degree of accuracy.

Also, in the embodiment of the present invention, the composition of the Ringer's solution to have the same osmotic pressure as that of human body fluid can prevent the Ringer's solution from penetrating into the skin of a human body. As a result, it effectively prevents the skin from swelling up by submerging it in the Ringer's solution through therapy, even in the case of prolonged exposure.

Also, the thermotherapy device in the embodiment of the present invention, since the Ringer's solution filled in the heat capsule 5 is drained off and thrown away without reuse, effectively prevents bacteria from breeding when bacteria outbreaks in case that the Ringer's solution is maintained at a predetermined high temperature. As a result, no problem occurs on sanitary conditions.

The embodiment of the present invention disclosed this time should be considered to be one of the examples and non-restrictive in every point. The claim of the present invention is revealed not by the above embodiment but claim itself. Moreover, all changes in claims and in the meaning of and within the equivalence to claim are included.

For example, in the above embodiment, the control computer 8 is constructed to control the temperature of the Ringer's solution. But, the present invention isn't restricted by this embodiment, and in the present invention, the control computer 8 may manage the human living body according to the situation of the human body at thermotherapy as well as controlling the temperature of the Ringer's solution.

Also, in the above embodiment, the example is revealed to apply the thermotherapy device to the cancer therapy. But, the present invention isn't restricted only to this, and the present invention can be widely applied to general thermotherapy to treat in use of the difference in temperature-sensitivity of healthy cells and other cells. For example, it can be applied to thermotherapy for viruses.

### INDUSTRIAL APPLICABILITY

As above, according to the present invention, almost the entire human body can be treated by thermotherapy. Also, the present invention takes effects to treat cancer cells that have metastasized to different parts of the body that are difficult to find. In addition, the present invention can regulate the temperature of a human body with a high degree of accuracy. The thermotherapy device in the present invention is produced in the manufacturing industry of thermotherapy devices, and is suitable for therapy.

1-12. (canceled)

13. A thermotherapy method comprising the steps of:
providing a container filled with a liquid and capable of submerging almost an entire human body except for the head in the liquid,
submerging almost an entire said human body except for the head in the liquid in the container,
giving said human body general anesthesia when said human body is submerged in the liquid in said container, and
controlling the temperature of the almost whole said human body to be at a predetermined temperature for therapy.

14. A thermotherapy method in claim 13 wherein said predetermined temperature for therapy is above 37 degrees centigrade and below 43 degrees centigrade.

15. A thermotherapy method in claim 13 wherein said predetermined temperature for therapy is above 40 degrees centigrade and below 41.5 degrees centigrade.

16. A thermotherapy method in claim 13 wherein the thermotherapy is performed at a rate of between 12 hours to 24 hours per cycle.
